(19) United States
(12) Patent Application Publication
(10) Pub. No.: US 2005/0182456 A1
(43) Pub. Date: Aug. 18, 2005

(54) Automated cortical mapping

(76) Inventors: John F. Ziobro, Delafield, WI (US); David A. Putz, Delafield, WI (US)

Correspondence Address: JANSSON, SHUPE & MUNGER, LTD, 245 MAIN STREET, RACINE, WI, 53403, US

(21) Appl. No.: 10/781,427

(22) Filed: Feb. 18, 2004

(51) Int. Cl. A61N 1/18

(52) U.S. Cl. 607/48

(57) ABSTRACT

A method for cortical mapping includes utilizing subdural electrodes as selectable stimulus points in a closed loop system of cortical mapping based on electromyographic detection events. A system for cortical mapping includes a plurality of subdural electrodes formed as a grid, a cortical stimulator for stimulating individual pairs of the plurality of subdural electrodes, and an electromyograph for detecting reaction to the stimulating. The system may include a controller structured for associating the reaction with one of the individual pairs of the plurality of subdural electrodes. The system and method allow quick and accurate functional mapping of an area of the brain, automatic mapping, and essentially realtime feedback for a probe localization process. Use of the system and method provides intraoperative functional localization while reducing a possibility of adverse effects to the patient.

### BACKGROUND

1. Field of the Invention

The invention relates to intraoperative brain stimulation and, more particularly, to stimulation of a cortex combined with electromyographic feedback.

2. Background of the Invention

Resectioning of brain tissue, for example surgical removal of epileptogenic tissue for treatment of medically refractory focal seizure disorders such as epilepsy, requires a high degree of accuracy, both in identifying the diseased cortex (e.g., an epileptogenic focus) as well as the viable cortex (e.g., a speech or motor function center). By accurately mapping a given area of the brain to an associated function, it is possible to minimize or avoid postoperative functional deficits.

Resection surgery may also include various procedures such as hemispherectomy, corticectomy, lobectomy and partial lobectomy. Less-radical procedures include lesionectomy, transection, and stereotactic ablation. In such procedures, there is a high risk of damage to functionally important brain regions and the consequent long-term impairment/destruction of various motor, cognitive and other neurological functions. It is therefore desirable to minimize damage to particular portions of the brain.

Various techniques may be used for general localization of brain function, and noninvasive methods such as positron emission tomography, regional cerebral blood flow, magnetoencephalography (MEG scans), Magnetic Resonance Imaging (MRI), and brain electrical activity mapping are known. MEG scans permit quantification of electrical activity in specific regions of the brain. Resolution of MEG scans has varying accuracy when the MEG scan is correlated with an MRI scan for pre-surgical purposes of identifying anatomical structures. Other techniques, for example, may focus on metabolic changes occurring in the extracellular milieu in response to neural activity and provide additional data. Such noninvasive procedures may lack the accuracy required for surgical precision, but provide a general mapping. In addition, noninvasive electrical and magnetic activity mapping may only provide a variable anatomical relationship due to underlying electrophysiological generators, confounded by differences in the spatial orientation of same.

For intraoperative functional localization in the brain during neurosurgery, for example a cortical resection, an invasive method may conventionally utilize electrocorticographic recording of electroencephalogram (EEG) and sensory evoked potentials, or a method may involve electrical stimulation of the brain. Conventionally, intraoperative localization of brain function using electrical stimulation may be implemented by using a cortical stimulator. A cortical stimulator is typically either a constant voltage or constant current device. The output from such a device may be directly applied as short current pulses of alternating polarity to different regions of the brain. A surgeon may stimulate areas of the brain using specialized probes or subdural electrodes and then observe effects of the stimulation on the patient such as by monitoring of EEG, by observing muscle twitching, or by observing obliteration of the ability to speak during stimulation of the language region. The surgeon uses this information to help guide the resection. An undesirable consequence of direct cortical stimulation is that seizures can be induced. In addition, such an electrical stimulation type mapping process takes a long time, which increases costs and trauma to a patient.

### OBJECTS OF THE INVENTION

It is an object of the invention to provide an improved method, system and apparatus for intraoperative localization of functional areas of the brain while overcoming some of the problems and shortcomings of the prior art, including those referred to above.

Another object of the invention is to provide a system and method for quickly and accurately obtaining a functional map of an area of the brain.

Another object of the invention is to provide a system and method for automatically obtaining a functional map of an area of the brain.

Still another object of the invention is to provide a system and method with essentially realtime feedback for a probe localization process.

Yet another object of the invention is to provide a system and method for intraoperative functional localization which reduces a possibility of adverse effects to the patient.

How these and other objects are accomplished will become apparent from the following descriptions and the drawings.

### SUMMARY OF THE INVENTION

Subdural electrode grids are commonly used in detection rather than for cortical stimulation. Methods exist for treatment of neurological disorders, such as by electrically stimulating the patient's vagus nerve, and for detecting neurological dysfunction. Such methods, for example, may be used for terminating epileptic seizures by providing electrical stimulation near the focus of the epileptogenic region, usually at the neocortex. This region is particularly susceptible to damage that may result in loss of speech, sensory disorders, and/or loss of motor function. Electroencephalogram (EEG) and electrocorticogram ECoG) waveforms are detected and used to help locate origins of epileptic activity, to minimize damage that could be caused by extraneous application of electrical stimuli. Subdural electrodes are conventionally used for such EEG/ECoG detection. The present inventors have determined that subdural electrodes may also be used as selectable stimulus points for a closed loop system of cortical mapping.

In an aspect of the invention, a system for cortical mapping includes a plurality of subdural electrodes formed as a grid, a cortical stimulator for stimulating individual pairs of the plurality of subdural electrodes, and an electromyograph for detecting reaction to the stimulating.

According to another aspect of the invention, a system for cortical mapping includes a plurality of subdural electrodes formed as a grid, a cortical stimulator for stimulating individual pairs of the plurality of subdural electrodes, an electromyograph for detecting reaction to the stimulating, and a controller structured for associating the reaction with one of the individual pairs of the plurality of subdural electrodes.

In a further aspect of the invention, a method of cortical mapping includes providing a plurality of subdural electrodes formed as a grid, electrically stimulating individual pairs of the plurality of subdural electrodes, detecting an electromyographic reaction to the stimulating, and associating the reaction with one of the individual pairs of the plurality of subdural electrodes.

According to an additional aspect of the invention, a method includes utilizing subdural electrodes as selectable stimulus points in a closed loop system of cortical mapping based on electromyographic detection events.

In a still further aspect of the invention, apparatus for intraoperatively localizing functional areas of the brain includes means for stimulating a portion of a cortex, means for detecting a reaction to the stimulating, and means for associating the detecting with the stimulating.

There is a need for automatically mapping different areas of the cortex in an efficient, safe and quick manner. By providing such a system and method, the invention allows for automated mapping at several different stages, for example, after placement of the subdural electrode grid, immediately before beginning a subsequent procedure, during the procedure, etc. Since the automated mapping may require only seconds or a few minutes total time, the mapping may be repeated many times. This allows a surgeon to accurately verify, for example, non-movement of the patient, determinations from previous mapping, etc. In some applications, the mapping may be implemented using continuous monitoring.

The foregoing summary does not limit the invention, which is instead defined by the attached claims.

### BRIEF DESCRIPTION OF THE DRAWINGS

FIG. 1 is a schematic diagram of a cortical mapping system according to an exemplary embodiment of the invention.

FIG. 2 is a more detailed schematic of a cortical mapping system according to an exemplary embodiment of the invention.

FIG. 3 shows an electrode strip used in a cortical mapping system according to an exemplary embodiment of the invention.

FIG. 4 shows an electrode grid used in a cortical mapping system according to an exemplary embodiment of the invention.

FIGS. 5 and 6 show different configurations for electrode arrays, such as dual-sided interhemispheric electrode arrays used in a cortical mapping system according to an exemplary embodiment of the invention.

FIGS. 7A-7D illustrate exemplary pulse trains produced by a cortical stimulator in an embodiment of the invention.

FIG. 8 shows a cortical mapping instrument according to an exemplary embodiment of the invention.

FIGS. 9A-9B show a front panel having two selectable control button formats, as used on the exemplary cortical mapping instrument of FIG. 8.

FIGS. 10A-10B show a flowchart for a mapping session according to an exemplary embodiment of the invention.

FIG. 11 shows a personal computer (PC) that may be used for monitoring and/or controlling a cortical mapping instrument according to an exemplary embodiment of the invention.

FIGS. 12A-12B show mono-polar type probes used in cortical mapping systems according to exemplary embodiments of the invention.

FIGS. 13A-13B show bi-polar type probes used in cortical mapping systems according to exemplary embodiments of the invention.

FIGS. 14A-14B show tri-polar type probes used in cortical mapping systems according to exemplary embodiments of the invention.

FIG. 15 shows a mono-polar type probe used in cortical mapping systems according to exemplary embodiments of the invention; the FIG. 15 probe may alternately be formed in a bi-polar or tri-polar configuration (not shown).

FIG. 16 shows a sphere electrode type probe used in cortical mapping systems according to exemplary embodiments of the invention.

### DETAILED DESCRIPTION OF PREFERRED EMBODIMENTS

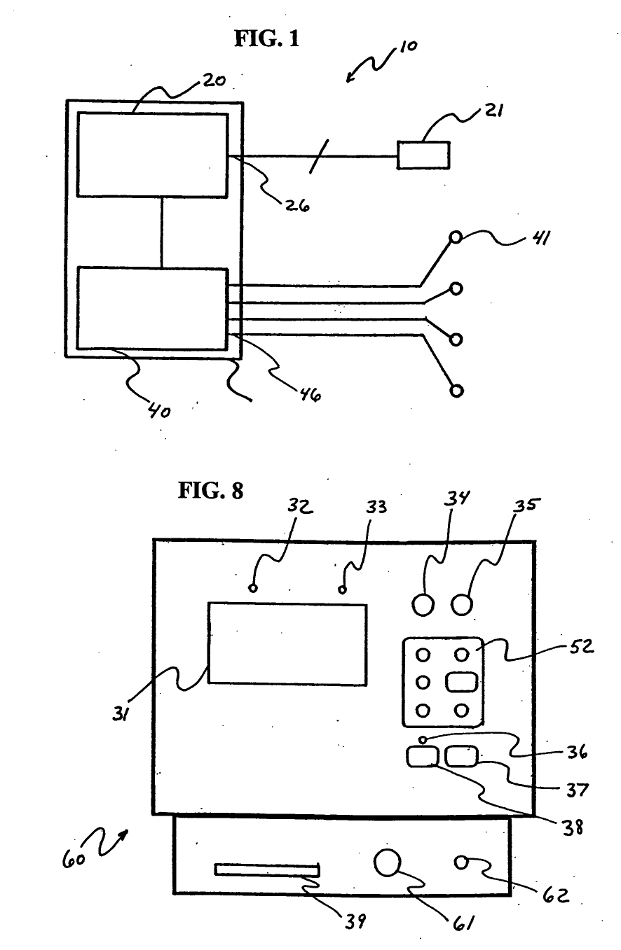

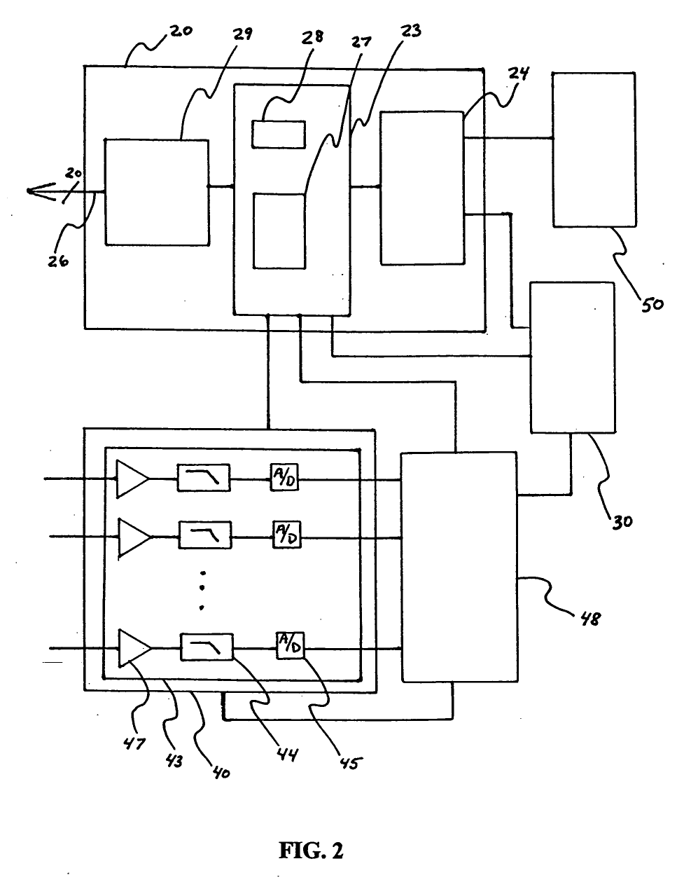

FIGS. 1 and 2 schematically show a mapping stimulator 10 for functional localization of an area of a brain according to an exemplary embodiment of the invention. The mapping stimulator 10 includes a cortical stimulator 20, a number of subdural electrodes 21 connected to corresponding output ports 26 of cortical stimulator 20, an electromyographic (EMG) detector 40, and a number of transducers 41 for detecting muscle activity and inputting corresponding action potential signals to input channels 46 of EMG 40.

For purposes of this invention, the terms “subdural” and “cortical” may be used interchangeably in describing electrodes used for cortical stimulating. In that regard, it is understood that any suitable electrodes may be utilized for effecting cortical mapping. Similarly, the particular form for a plurality of electrode contacts may variously be described as “strips” or “grids” and it is understood that the present invention contemplates that such form may be chosen as one suited for a particular application. Therefore, while these terms may be equivalent in certain respects, they are not required to be so.

Cortical stimulator 20 provides electrical signals to the plurality of subdural electrodes 21 placed on the cortex of a patient, via a number of output ports 26 that varies depending on the configuration of electrodes 21 and associated interfacing. Muscle activity of the patient is monitored by EMG sensors 41 that provide detection signals to a signal conditioning and processing section 43 in the input portion of EMG 40. The EMG signals are filtered and digitized for further analysis. Signal processor 43 automatically determines whether a stimulus applied to a pair of the electrodes 21 has caused a muscle reaction, by detecting a muscle contraction in the signal(s) from sensors 41 and correlating the detected contraction with a particular stimulus. The correlation may be presented graphically to a surgeon as a map of functions of particular areas of the cortex.

Cortical stimulator section 20 of mapping stimulator 10 provides high-precision output pulses for cortical mapping. A CPU 27 of controller 23 determines voltages to be applied to individual pairs of electrode contacts 82 (FIG. 4) and a sequence to be used for a given stimulation pass. The voltages and sequence(s) may be specified by a user, or may be calculated by CPU 27 based on feedback, predetermined patterns, and algorithms such as those employing known step functions and the like. Cortical stimulator 20 contains a signal generator 29 operative to output stimulation signals and selectively change various parameters thereof. CPU 27 operates in conjunction with a clock 28, so that timing of stimulation signals may be accurately matched with a subsequent EMG detection event. Clock 28 may be implemented as multiple clocks and/or may be used in conjunction with one or more external clocks such as a system clock. Signal generator 29 outputs separate pulse trains to individual pairs of the electrodes 82. Variable parameters of the pulse trains output by signal generator 29 are controlled by CPU 27, including a number of pulses in a pulse train, voltage, current, and pulse width of individual pulses, frequency, time between consecutive pulse trains, polarity, waveform rise and decay, and associated filtering. Multiplexing may be used in signal generator 29. In various embodiments, stimulation parameters may be adjusted and/or set and implemented in hardware or software.

Cortical stimulators may be either constant voltage or constant current devices used intraoperatively to localize motor and/or language centers. Traditionally, a surgeon stimulates areas of the brain and observes whether muscles move and/or speech is affected. Although research (e.g., Calancie, et al.) has quantified specific relationships between parameters of an applied stimulus voltage and an evoked muscle response, the present inventors have determined that such conventional methods do not differentiate between several different points of stimulation, and do not consider a use of multiple pairs of stimulating electrodes such as for mapping individual areas. Both depth electrodes and subdural electrodes may be used. Depth electrodes penetrate deep into the brain tissue in direct contact with such tissue, while subdural strip or grid electrodes are placed beneath the dura in direct contact with brain tissue at the surface of the brain without penetrating brain tissue.

Cortical stimulator 20 may operate on either AC line voltage and/or by use of a rechargeable battery, for example a battery that provides eight hours continuous use and that is able to be completely recharged in sixteen hours. Such a battery preferably has an overcharge protection circuit. Cortical stimulator may be provided either as a constant current device or as a variable voltage device. When formed as a constant current device, cortical stimulator 20, for example, may have an stimulation output adjustable between 0-36 mA, peak-to-peak, in 0.1 mA increments. Other selectable ranges may include, for example, 0-10 mA, 0-1 mA, 0-100 μA, etc. Adjustment resolution may be as small as, for example, 1 μA. Adjustments in the output current may be implemented as ‘slow’ or ‘fast’ adjustments, or by using a simple step function. The output may be a +/−square wave with a pulse duration that is adjustable, for example, from 10 μsec to 3.0 msec/phase. Maximum voltage in the constant current mode is typically set to 100 volts. Pulse frequency may be adjusted, for example, from one to one thousand hertz in one hertz increments. Pulse trains may be formed for durations, for example, of from 1 msec to 10 seconds. A ‘stimulation active’ indicator 33 is lighted while pulse trains are being output via port(s) 26.

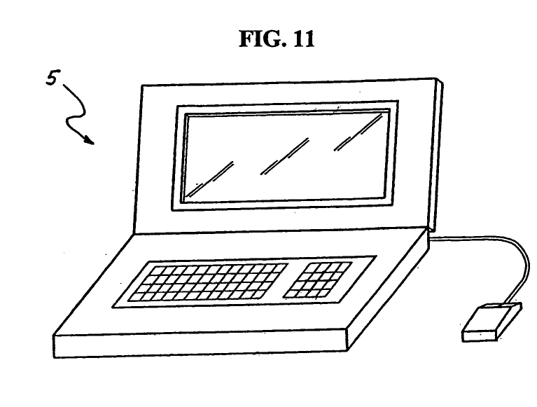

When formed as a variable voltage device, cortical stimulator 20, for example, may have an output with a voltage that is adjustable for each pulse from 0 to 800 volts, adjustable via software control using an attached PC 5 (FIG. 11) or in a manual mode by use of multi-turn knobs 34, 35 (FIG. 8). Pulse width for such pulses may be adjusted, for example, from 10 μsec to 3.0 msec/phase, where a pulse width of approximately 0.4-0.6 msec may be selected as a nominal value. Pulse trains may be formed for one to twenty pulses, with 0.1 msec to 4 msec between adjacent pulses being typical. Current limiting circuitry may be utilized for safety and may include short circuit protection and the like. Load impedance for attached electrode arrays 21 may include high precision standard resistance of individual electrode loops between electrode pairs, and/or may be adjusted within the cortical stimulator section 20 as an output impedance. Polarity reversing switch circuitry, offsetting circuitry or voltage conversion circuitry, etc. may be integrated in signal generator/multiplexer 29 for providing alternating polarity pulse trains to outputs 26.

An exemplary EMG system 40 detects the electrical response of a muscle using pairs of noninsulated electroencephalography-type needle electrodes 41 that are placed subcutaneously overlying each target muscle. Alternatively, surface type electrodes, monopolar or bipolar EMG type needle electrodes, and others may be used for EMG detection. In EMG section 40, the response signals from pairs of sensing electrodes 41 are amplified, filtered, and processed by being buffered, digitized, and placed in a memory space 30.

EMG detector 40 is used for detecting electromyographic events from monitored EMG signals. EMG signals are small bioelectrical signals associated with nerve and muscle activity. EMG detector 40 receives biphasic and/or monophasic action potentials from individual sensors 41. For example, a finger pulse transducer or other pressure pad type sensor, and others such as nerve conduction type transducers may be used to detect nerve and muscle activity. The received signals are input to input module 43 having small signal amplifiers 47 and filters 44 for input channels 46. The input channels 46 of the input module 43 are preferably impedance matched with the sensors 41 and have analog-to-digital (ADC) conversion of the received signals for further processing. A high sampling frequency is preferably chosen for the ADC 45 for obtaining high accuracy digital signals for each channel, although a conventional sampling rate of approximately 2 kHz may alternatively be used. The digital signals may be stored in memory 30, which is adapted for quasi realtime processing such as time stamping of data, high accuracy filtering, etc. A high performance processor 23 and high capacity memory 30, along with high accuracy digital signal processing (DSP) schemes implemented in a DSP 48, are used for reducing quantization and other effects of the ADC 45. In addition, the amplifier stage(s) 47 are optimized for obtaining an analog voltage swing that achieves high accuracy of quantization while also obtaining high noise immunity, and for obtaining linear operation.

Careful design of the input module 43 by optimizing gain and bandwidth considerations avoids line interference, motion or stimulus artifact, and other system noise. The processing of the received signals may include Fourier type transformation, anti-aliasing, bandwidth reduction, noise identification and adaptive filtering, phase distortion identification and elimination algorithms, and others. Various analog and digital filter types may be used, such as multi-stage analog filtering (signal conditioning) prior to ADCs 45, and in subsequent DSP 48. In an exemplary embodiment, muscles activities may be monitored as surface EMG signals and recorded from bipolar Ag/AgCl electrodes available from NEC Medical Systems, Japan. Such electrodes may be connected to a preamplifier (not shown) and a differential amplifier 47 having a bandwidth of 5 Hz to 1 kHz and having a part number 1,253A, also available from NEC. The EMG signals may be collected with a sampling frequency of approximately 1 kHz. The analog EMG signals may be high-pass filtered at 20 Hz, low-pass filtered at 500 Hz using 4th-order digital Butterworth filters, full-wave rectified, and smoothed with the use of a bidirectional digital low-pass Butterworth filter with a 2-Hz cut-off frequency, to yield smoothed rectified EMG. Digital filtering may or may not include feedback processing (recursive filters). Further, by ‘processing-out’ baseline noise to prevent saturation and account for high noise artifacts, just prior to taking EMG measurements, a high accuracy is achieved. By reducing baseline noise to a minimum, an extremely low EMG signal may be detected and quantified. Optionally, reference sensors may be utilized, including a ground electrode inserted subcutaneously between stimulating electrode contacts 21 and recording sensors 41, a reference sensor inserted into a heel pad, and a recording sensor inserted into the plantar muscles of the foot. Resulting compound muscle action potentials (CMAPs) and the like may be recorded and processed to determine, for example, their negative wave peak value, and corresponding normalization values.

In the EMG detection portion 40, signal processor 43 receives the detection signals from sensors 41, either on individual channels as separate dedicated channels or by being selected for a given channel such as by using time-division multiplexing. The detection signals are fed to differential amplifiers 47, each receiving analog signals from a corresponding pair of bipolar EMG electrodes 41 and outputting a respective amplified signal at a level suitable for subsequent processing. Anti-aliasing and other low pass filters 44 (e.g., noise filtering) are preferably used in signal conditioning portion 42 of the analog input section. Exemplary input filtering controls may include selectable sensitivity settings from approximately 2 to 500 μV/division, low-pass 2-pole 12 dB/octave filters with selectable cutoff frequencies, high-pass 1-pole filters with selectable cutoff frequencies, notch filters such as for eliminating line noise, sensor temperature compensation, and impedance matching.

The amplified and filtered signals are fed to analog-to-digital converters (ADC) 45 that each output a digital signal corresponding to the respective bipolar signal. The digital signals are then fed to a digital signal processor (DSP) 48, either directly or via a buffer or memory 30. Artifact rejection may be adjusted for each channel. The digital data may be filtered, multiplexed, interleaved, channelized, exported, etc. by DSP 48. Digital filtering may be modified according to a particular application such as by defining a number of taps and/or a memory size for recursive filtering, selecting various parameters based on baseline waveform responses, etc. Signal processor 43 and cortical stimulator 20 are controlled by a CPU 23.

The ADCs 45 preferably use a sampling frequency several times the frequency of the analog signals in order to further reduce aliasing. The sampling rate of an ADC 45 may be changed depending on available memory space and other processing considerations such as resolution for quantization of data. For example, a higher resolution may be obtained by using an ADC 45 with a higher number of quantization bits, although this may be limited by cost considerations. The DSP 48 may use Fourier transformation for representing an EMG signal as a sum of weighted sinusoids. The superposition of the several component potentials acts to eliminate phase distortion when appropriate phase characteristics are implemented in subsequent filters. This is important because slight changes in muscle fiber orientation, motor unit firing rates, and electrode contact position may cause significant changes in phase characteristics. It is possible to utilize this EMG phase information for isolating such effects, but it is generally more appropriate to eliminate phase characteristics and derive amplitude response characteristics. A Fast Fourier Transformation (FFT) may be used for extracting frequency information from the EMG signals during the digital signal processing.

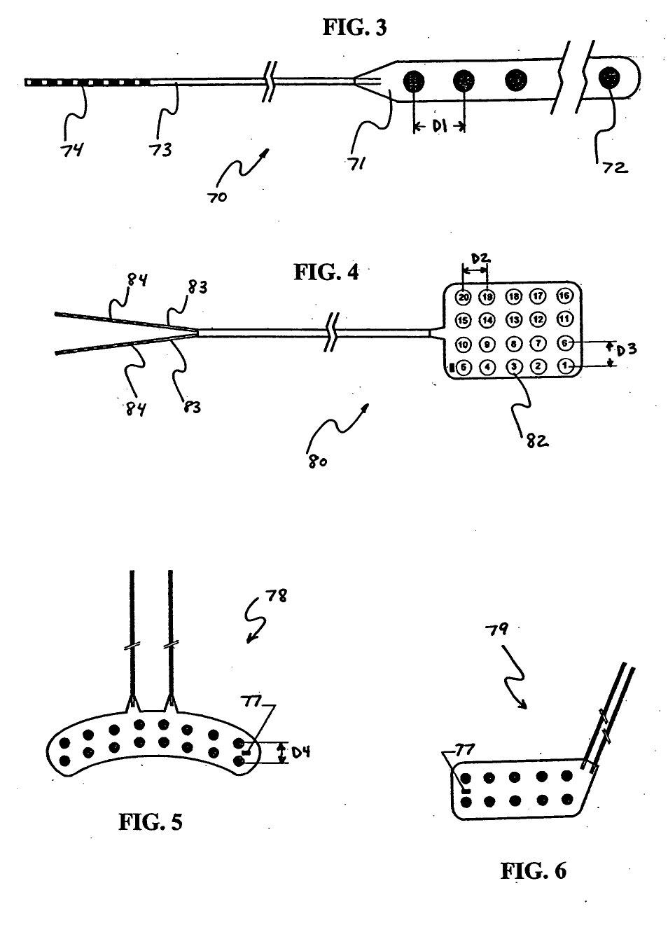

FIG. 3 shows a subdural electrode strip 70 for use in mapping stimulator 10. For example, subdural electrode strip 70 may be as disclosed in U.S. Pat. No. 6,004,262, granted to Putz et al. and herein incorporated by reference. The relative safety of subdural strip electrodes lies in the fact that, unlike depth electrodes, they are not invasive of brain tissue. By comparison, depth electrodes are narrow, typically cylindrical dielectric structures with contact bands spaced along their lengths. Depth electrodes are inserted into the brain in order to establish good electrical contact with different portions of the brain. Subdural strip electrodes, on the other hand, are flat strips supporting contacts spaced along their lengths. Such strip electrodes are inserted between the dura and the brain, along the surface of and in contact with the brain, but not within the brain. Such a subdural strip electrode assembly 70 has an elongated flexible dielectric strip 71 within which a plurality of spaced aligned flat contacts 72 and their lead wires are enclosed and supported in place, sandwiched between front and back layers of material forming the dielectric strip 71. Each flat contact 72 has a face or main contact surface which is exposed by an opening in the front layer of the dielectric strip. The dielectric material used in such subdural strip electrodes is a flexible, medically-acceptable material such as silicone. Contacts may be formed of material such as gold or platinum though, as is recognized in the art, any conductive corrosion-resistant and non-toxic material may be used.

Electrode strip 70 has a tail portion formed of a small-diameter, elongate, cylindrical, flaccid, flexible, electrically insulating material such as a silicone material or a polyurethane as the tail body 73. The body 73 has collar-like, tubular electric contacts 74 closely fitted around its outside surface. Each contact 74 is permanently attached to a separate insulated wire (not shown) that extends from the contact 74 through the body 73 to the respective electrode 72. The electrodes 72 may be formed of platinum, stainless steel, or other appropriate conductive material. Spacing between adjacent electrodes 72 (i.e., center-contact to center-contact) may be chosen in general in a range from about 2 to 15 mm. For example, a standard 10 mm spacing D1 between adjacent electrodes 72 may be adequate or, alternatively, a particular spacing between adjacent electrodes 72 may be customized for a particular application such as for different size cortex or for different resectioning operations, etc. Similarly, a diameter of individual electrodes 72 may be chosen in a range from about 0.5 to 10 mm. A 4-6 mm size with a corresponding 2-4 mm of exposure is typical. Subdural electrode strip 70 is characterized in that it provides advantages by being transparent, thin, flexible, and available in a variety of different sizes. Tails 73 of electrode strips 70 are typically either 1.5 mm or 2 mm in diameter. The latter may be used in standard DIN type connectors.

FIG. 4 shows a subdural electrode grid 80 formed as an array of electrodes 82. In this example, a four by five grid is chosen, but the arrangement and number of electrodes 82 in a grid 80 may be chosen for a particular application. For example, various subdural electrode grids are available from Ad-Tech Medical Instrument Corporation of Racine, Wis. A number of tails 83 depends on the electrode configuration. Here, subdural electrode grid 80 has two tails 83 of ten contacts 84 each. Contact spacings D2, D3, respectively for columns and rows of contacts 84, are typically each 10 mm, but any suitable array spacings may be used. Individual electrode discs 82 may be physically numbered for assisting the physician intraoperatively. An electrode grid may be formed in any configuration including three-dimensional. For example, a three-dimensional grid may be formed by using two or more individual electrode strips 70 or electrode grids 80, or may be a unitary structure.

FIGS. 5 and 6 respectively show dual-sided interhemispheric electrode arrays 78, 79 that are specially adapted, for example, for placement in a fissure of the brain. Such electrode placement may be utilized with the present invention. Electrode arrays 78, 79 are formed of two individual electrode arrays paired uniformly together back-to-back. Electrode arrays 78, 79, for example, may be approximately 1.0 mm thick, with electrode contacts 4.0 mm in diameter having 2.3 mm exposures. Contact spacing D4 is typically 10 mm, although any desired spacing may be used. The number of contacts may be selected according to different configurations and corresponding array formats. A marker 77 may be located on one side of the array structure, to assist in operative procedures involving, for example, location or orientation of the marker 77.

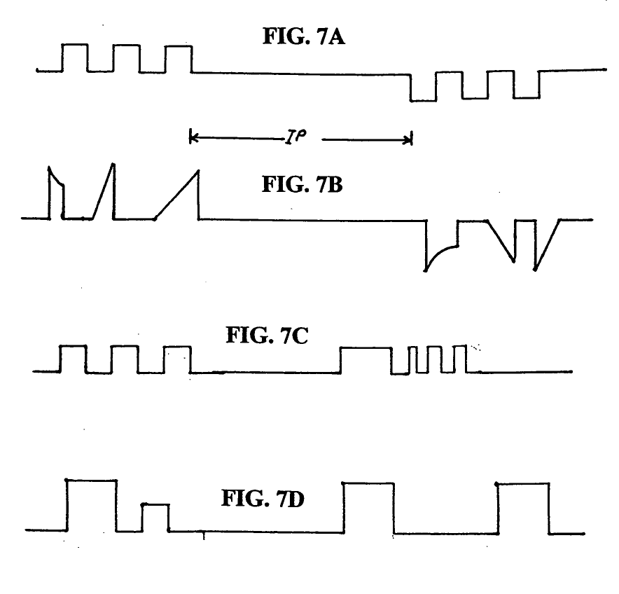

Various exemplary pulse trains are shown in FIGS. 7A-D. For example, FIG. 7A shows two separate pulse trains each having three individual pulses that are square waves of a same magnitude and pulse width. The consecutive pulse trains in this example have reversed polarities. A time between pulse trains is denoted by the letters “IP” for interpulse time. By comparison, FIG. 7B is an example of using different waveforms for individual pulses. Specifically, a first pulse in each pulse train has a fast rise time and a much longer fall time, whereas the second and third pulse of each pulse train each have a sawtooth wave shape. As in the previous example, the consecutive pulse trains have opposite polarities. The example of FIG. 7C illustrates a first pulse train having three pulses with the same pulse width, followed by a second pulse train composed of four pulses where the second, third, and fourth pulse of the second pulse train each have a pulse width that is narrower than the first pulse of the second pulse train. FIG. 7D is an example illustrating how individual pulses in a pulse train may each have a different amplitude and/or pulse width, and may also have different times between the individual pulses. In the last two examples, the polarities of consecutive pulse trains are shown to be the same, although any combination of variable parameters may be implemented for the pulses being output by cortical stimulator 20.

FIG. 8 shows an exemplary cortical mapping instrument 60 that provides multifunctional control and display capabilities in a portable and lightweight unit. Alternatively, a known cortical stimulator, such as a Nimbus model available from NewMedic in Toulouse, France, may be adapted for use with various aspects of the present invention. Referring back to FIG. 8, the exemplary cortical mapping instrument 60 has an LCD screen 31 that provides a large-size display area while consuming relatively little power at low voltages. LCD screen 31 may be a high resolution tft type display, a touch screen, and/or provided as an external screen. Power to instrument 60 is provided by a low noise AC power supply with an internal battery backup. A low battery indicator 32 is provided for monitoring the voltage and/or power state of a rechargeable battery pack. Instrument 60 has a cortical stimulator 20 that provides stimulation pulse trains to a probe output module 39 that is adaptable to accept leads, tails, wires, and various connectors for monopolar straight electrodes, bipolar straight electrodes, bipolar angled electrodes, bipolar straight sheathed electrodes, bipolar Y-tip electrodes, tripolar straight electrodes, tripolar angled electrodes, one, two or three-dimensional electrode arrays, ball electrodes, etc. Such adaptation may be provided by use of plug-in adapters (not shown).

Remote control capability and data exchange are provided by use of a remote control input/output port 61 that may use known technologies such as hardwired connectors, fiber optic connectors, IR port, USB, firewire, etc. Such remote control capability may be independent of a use of a remote PC such as a laptop 5 shown in FIG. 11. For example, a PC may be implemented for data logging, pattern modification, mapping analysis, etc, while simultaneously providing a physician or technician with the ability to trigger individual mapping passes or updated results from a remote control device. Connection of instrument 60 to PC 5 may be effected using known serial, parallel, intranet, Internet, etc., and may employ various applications and protocols such as ftp, smtp, and the like. Additionally, remote control input/output port 61 may be adapted for communicating with a simple IR type wireless remote control device, such as for implementing simple on/off stimulation control for individual stimulation passes. A separate remote on/off button 38 and indicator light 36 may be used on instrument 60, respectively, for enabling/disabling remote control operation and showing a status of same. For example, a tri-state LED may be used as indicator light 36, so that additional features may be implemented for remote control status.

Cortical stimulator 20 may alternatively be operated in a manual mode where individual stimulation parameters such as an individual output level for pulses may be controlled using front panel controls. The manual mode may also be used for inputting individual steps of a stimulation routine and the corresponding parameters, as discussed further below. As shown by example in FIG. 8A, front panel 52 is also adapted to select preloaded stimulation routines and corresponding parameters from a menu displayed on LCD 31. In such a case, a surgeon can select from factory settings and from programs she has previously loaded into the menu by either manually inputting the settings or by downloading the routines from a remote computer. Cortical stimulator 10 may alternatively be operated in a remote operations mode, such as by being connected to a computer, for example a laptop PC (not shown).

Predefined stimulation patterns may be selected using parameter controls 53, 54, 55 in response to prompts being displayed on LCD 31 or on the display of the attached PC. For example, a user may start with a base map selected from a menu, and then modify the default parameters of the base map. The base map has a number of parameters for a stimulation event. The stimulation event contains a number of individual stimulations and times between the stimulations. A type of customizing of a base map may involve adjusting for a latency of feedback from an EMG sensor, increasing an amplitude of a stimulation for individual electrode contacts 82, utilizing previous mapping results and/or patient neurological data, utilizing physiological data, and/or other modifications to achieve a high accuracy starting point. A use of concurrent data for optimizing the base map may include using recurrence plot strategies and various methods for deriving univariate or multivariate measures that characterize the deterministic and/or diagnostic structure in the signals being analyzed, thereby providing a basis for discrimination.

A first stimulation may be programmed to have precise settings for ramping rate, peak voltage, pulse width, decay rate, pulse shape, and overshoot amount for a first pulse. The first stimulation has an adjustable time between the first pulse and a second pulse, and the second pulse of the first stimulation may have its waveform parameters uniquely set in a manner as described for the first pulse. Alternatively, all the pulses in a stimulation event may have a same waveform, whereby a user may only be required to set the number of pulses, duty cycle, frequency, and amplitude. When individual time delays between adjacent pulses of a stimulation are not being manually programmed, a frequency for repetition of pulses within a stimulation may be programmed by the user.

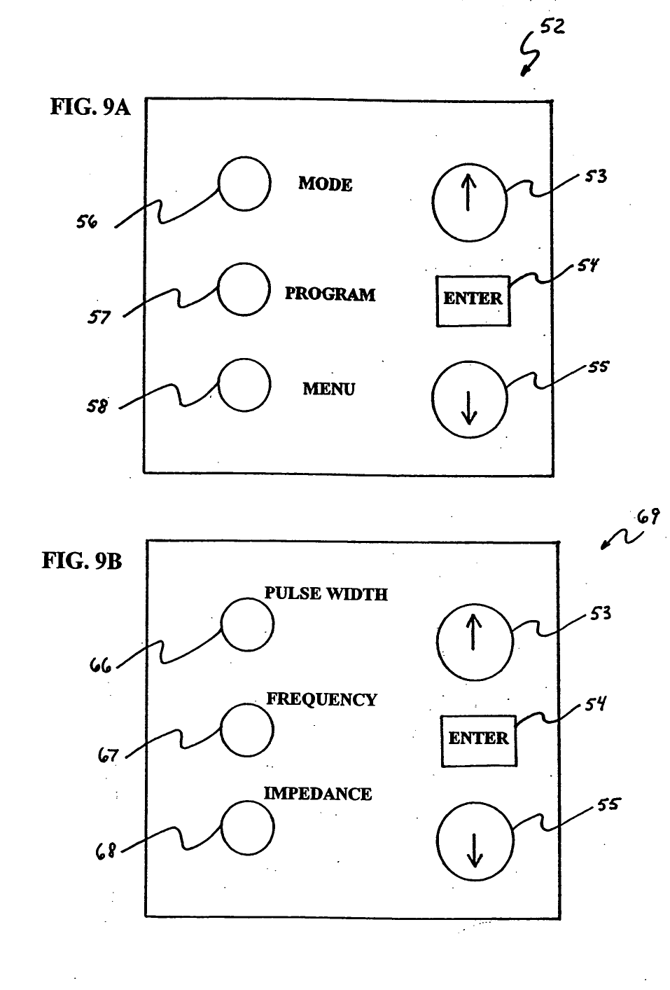

As shown by example in FIGS. 9A-B, various buttons of a front panel area may be selected as either an automatic control front panel 52 or as a step control front panel 69. For example, firmware allows switching between front panel 52 and front panel 69 by pressing and simultaneously holding down buttons 53 and 55 for a time greater than three seconds. When front panel 52 has been selected, a graphic is illuminated for each of the words “MODE”, “PROGRAM”, and “MENU” by backlighting corresponding areas on a back side of the front panel area. When front panel 69 has been selected, the backlighting changes to provide a graphic that illuminates the words “PULSE WIDTH”, “FREQUENCY”, and “IMPEDANCE”. Various controls of cortical mapping instrument 60 may be activated using front panel 69 for either manual programming of individual settings for an automated stimulation pass or for modifying parameters of individual stimulations having one or more pulses or pulse trains. For example, when a manual stimulation button 37 is depressed, the controls of front panel 69 are enabled for adjusting the parameters for a pulse, pulse train, or series of simulation events currently being indicated on LCD 31 for a next simulation. When it is desired to change the selection of the pattern to be modified, the buttons 66, 67, 68 may be un-selected again by switching back to front panel 69, which enables a selection menu to be displayed on LCD 31 and to be changed by using buttons 53, 54, 55. Since it is important to assure that pulse parameters are not inadvertently changed in error, the firmware automatically changes the front panel area back to front panel 52, as a default screen, if there has been no operational activity for a period of time such as one minute. Similarly, a ‘lockout’ may be selected in the ‘run’ mode to assure that a stimulation session is not inadvertently interrupted by an accidental pressing of a front panel button. Conversely, a ‘stop simulation’ button 62 is provided in a recessed portion of cortical mapping instrument 60 as a mechanism for immediately stopping a stimulation routine. Such provides for halting a current mapping session, for example in an emergency or during a multiple-pass mapping session.

In operation of front panel 69, pulse width is modified from a default setting by pressing button 66, then adjusting using buttons 53 and 55. When a desired pulse width value is displayed on LCD 31, the enter button 54 is pressed to set the pulse width value for the currently indicated pulse or set of pulses. Such also returns LCD 31 to the simulation event selection menu screen. The frequency for a currently selected series of pulses or pulse trains may be set by pressing the frequency button 67, then adjusting using buttons 53 and 55. When a desired frequency value is displayed on LCD 31, the enter button 54 is pressed to set the frequency value for the currently indicated pulse train or set of pulse trains. Such also returns LCD 31 to the simulation event selection menu screen.

An impedance check may be performed for the electrodes 21 connected to cortical mapping instrument 60, by pressing an impedance test button 68. The impedance test implements a known current loop test for each of the electrodes. Such will detect the presence of a short or open circuit, and verifies proper connection of tails 73, 83 with output 39.

Pulse trains are applied to individual pairs of electrodes 21 as individual simulation events. For example, it is desirable to minimize a voltage applied to a given area in order to minimize damage from the stimulus such as by reducing the possibility of inducing seizures (See, e.g., Calancie et al., Threshold-level repetitive transcranial electrical stimulation for intraoperative monitoring of central motor conduction, J Neurosurg 95: 161-168 (2001)). Cortical stimulator 20 has an assortment of algorithms for pragmatically and methodically increasing stimulus voltages only to the degree necessary for producing an EMG response. The EMG response is, generally, an ‘all-or-nothing’ type detection event. For example, in an axonal spike, the rapid wave of depolarizing current travels down an axon membrane and causes neurotransmitters to be released to a next post-synaptic space. As a result, a detectable electromyographic event is not greatly enhanced by applying additional voltage in a stimulus, beyond a minimum stimulus voltage required to produce an EMG response. As noted above, damage and/or seizures may result from applying too large a stimulus. Patterns for reversing polarity of stimulus pulse trains may be developed in conjunction with algorithms for minimizing stimulation voltages, in order to account for ion transfer properties and phenomena of physiological changes induced by the stimulation itself. In addition, stimulation patterns may be dynamically modified based on coincidence detection and other feedback, such as expert systems, modeling functions, sensory and physiological monitoring and analysis, response patterns, etc.

In addition, detecting a combined or other threshold EMG effect may include a use of advanced methods such as those disclosed in U.S. Pat. No. 6,547,746 granted to Marino, herein incorporated by reference, which may add to a confidence level for a detected event and may possibly lower a detection threshold. In most cases, conventional EMG monitoring may be used for detection of muscle response to stimuli.

A mapping session may be separated into implementations of a series of stimulation patterns. For example, a baseline mapping of patient neurological status may be obtained prior to surgery by comparison of EMG measurements (e.g., from voluntary movement) to average values of a population of like patients, consideration of patient's age and general health, data from previous cortical stimulation testing of like patients, etc. The baseline mapping is used for setting voltages to be applied to individual pairs of electrodes 21 during a first pass of stimulation. Additional factors that are considered when setting the voltage level(s) include the type of anesthetic used during surgery, time of anesthetization, type of surgery, etc.

Before beginning the mapping session, the mapping stimulator 10 is placed in an appropriate position and associated subdural/cortical strip electrode grid 70 or grid 80 is in place along the desired portion of the patient's brain in a known manner. For example, an incision may be made in the scalp over the site of proposed electrode placement. For example, strip electrodes are commonly placed through standard burr holes, while grid electrodes typically require a craniotomy. In either case, an incision may be made in the dura across the diameter of the opening. Electrode grid 80 may be moistened and its edge grasped with forceps. A Penfield dissector or similar implement is used to help pass electrode grid 80 under the dural edge. The grid 80 may be pushed into the space between the dura and the brain until it is completely inserted between the dura and the brain, oriented such that exposed contact discs 82 are on the side of grid 80 in contact with the surface of the brain.

Optionally, tunneling (passing) needles (not shown) may be used for tunneling the wire carriers away from the surgical site. For example, subdural strip electrodes and methods of their placement are described in U.S. Pat. No. 4,735,208 granted to Wyler et al., herein incorporated by reference. A passing needle is available from Ad-Tech of Racine, Wis. In a manner similar to that described in the '208 patent, wires or tails 73 for each electrode strip 70, or for a grid point pathway 83 that includes a row contact and a column contact, may be brought out through the skin by first threading them through the needle and then drawing them through the scalp at a distance from the burr hole incision.

It is noted that most brain mapping procedures are done intraoperatively. It is also possible, however, to install subdural/cortical electrodes in other situations where cortical mapping is desired. For example, after correct placement of the subdural grid 80 is confirmed, the scalp may be closed in layers and a dressing applied over the burr hole/craniotomy incision. Subsequent removal of subdural electrode grid 80 at a later time may include reopening the burr hole/craniotomy incision and reopening the dura incision. In such a case, the wires/tails are typically cut at a location near the proximal end of the electrode grid 80 and removed by outward movement through the needle wounds in the scalp. The body of the electrode grid 80 may then be grasped with forceps and removed through the reopened burr hole/craniotomy incision. The incisions may then reclosed and appropriate dressings applied to both the reclosed burr hole/craniotomy incision and needle wound or wounds.

EMG sensors 41 are then installed either as surface electrodes or subcutaneously in a known manner. For example, monitoring sensors and methods may be used such as those described in U.S. Pat. No. 6,654,634, granted to Prass and incorporated herein by reference. EMG sensors 41 are electrically attached to EMG processing section 40. After installation of EMG sensors 41, it is necessary to calibrate EMG sensors 41 to establish a baseline and to process-out the ambient noise using adaptive filters. A sensor setup may include use of a ‘third wire’ as an input for calibrating with a known calibration signal. In addition, the above-mentioned impedance check may be performed to assure there are no shorts or open circuits in the sensor circuits.

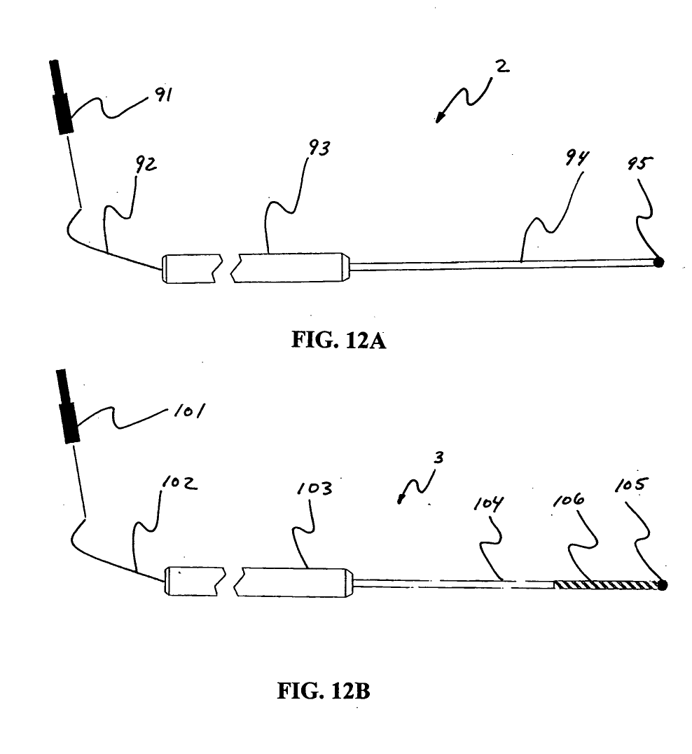

Various types of probes may be used as EMG sensors. For example, FIGS. 12A-16 illustrate exemplary mono-polar, bi-polar, and tri-polar type probes, as well as a sphere type electrode, any of which may be selected for particular applications. The Probes may be either rigid or flexible, with straight shafts or angled shafts, and with spring-loaded tips or regular tips. Preferably, each type of probe is disposable and not suitable for re-use. In FIG. 12A, a mono-polar probe 2 has a single conductor cable 92, for example 2 meters in length, that terminates with a safety plug 91. Cable 92 extends from one end of plastic handle 93, for example having a diameter of 10 mm and a length of 120 mm. A bendable or rigid, insulated, conductive metal shaft 94, for example having a diameter of 1 mm, extends from the other end of handle 93 and has an exposed conductive sphere tip 95. In FIG. 12B, a mono-polar probe 3 has a single conductor cable 102, for example 2 meters in length, that terminates with a safety plug 101. Cable 102 extends from one end of plastic handle 103, for example having a diameter of 10 mm and a length of 120 mm. A bendable or rigid, insulated, conductive metal shaft 104, for example having a diameter of 1 mm, extends from the other end of handle 103 and has an exposed conductive sphere tip 105 that has an insulated coiled spring 106 for greater flexibility.

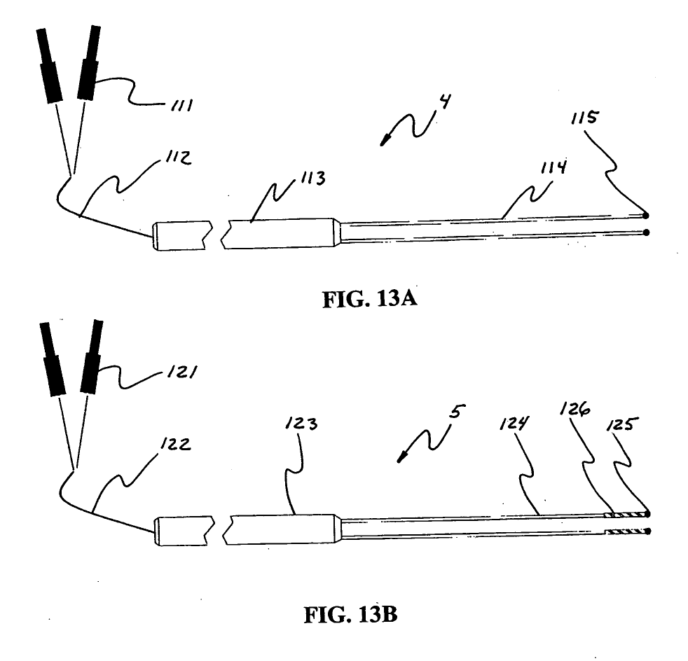

In FIG. 13A, a mono-polar probe 4 has a two conductor cable 112, for example 2 meters in length, that terminates with a safety plug 111. Cable 112 extends from one end of plastic handle 113, for example having a diameter of 10 mm and a length of 120 mm. Two bendable or rigid, insulated, conductive metal shafts 114, for example having a diameter of 1 mm, each extend from the other end of handle 113 and have an exposed conductive sphere tip 115. In FIG. 13B, a mono-polar probe 5 has a two conductor cable 122, for example 2 meters in length, that terminates with a safety plug 121. Cable 122 extends from one end of plastic handle 123, for example having a diameter of 10 mm and a length of 120 mm. Two bendable or rigid, insulated, conductive metal shafts 124, for example having a diameter of 1 mm, each extend from the other end of handle 123 and have an exposed conductive sphere tip 125 that has an insulated coiled spring 126 for greater flexibility.

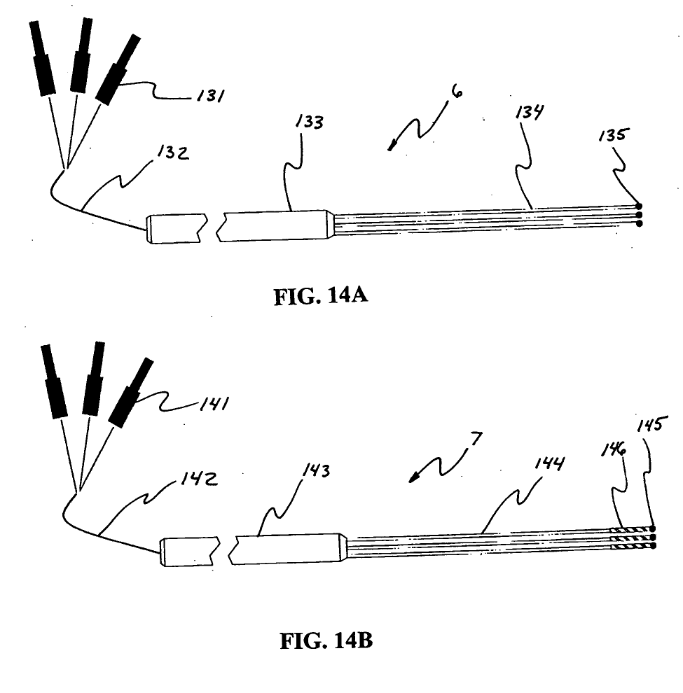

In FIG. 14A, a mono-polar probe 6 has a three conductor cable 132, for example 2 meters in length, that terminates with a safety plug 131. Cable 132 extends from one end of plastic handle 133, for example having a diameter of 10 mm and a length of 120 mm. Three bendable or rigid, insulated, conductive metal shafts 134, for example having a diameter of 1 mm, each extend from the other end of handle 133 and have an exposed conductive sphere tip 135. In FIG. 14B, a mono-polar probe 7 has a three conductor cable 142, for example 2 meters in length, that terminates with a safety plug 141. Cable 142 extends from one end of plastic handle 143, for example having a diameter of 10 mm and a length of 120 mm. Three bendable or rigid, insulated, conductive metal shafts 144, for example having a diameter of 1 mm, each extend from the other end of handle 143 and have an exposed conductive sphere tip 145 that has an insulated coiled spring 146 for greater flexibility.

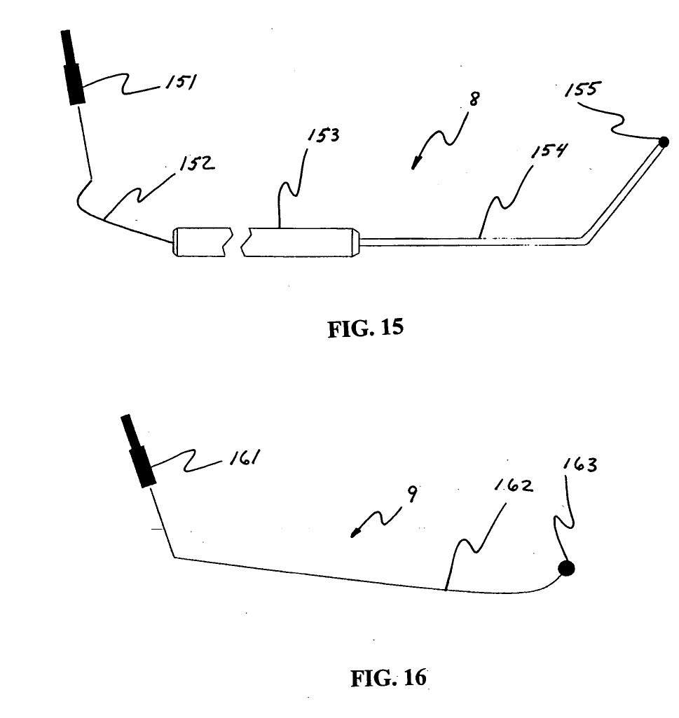

FIG. 15 shows a disposable type probe that may be formed in any of a mono-, bi-, or tri-polar configuration similar to the configurations of FIGS. 12A-14B described above. In FIG. 15, a mono-, bi-, or tri-polar probe 8 has, respectively, a mono-, bi-, or tri-conductor cable 152, for example 2 meters in length, that terminates with a safety plug 151. Cable 152 extends from one end of plastic handle 153, for example having a diameter of 10 mm and a length of 120 mm. Respectively, one, two, or three rigid, insulated, conductive metal shafts 154, for example having a diameter of 1 mm, each extend from the other end of handle 153 with a fixed angle shaft style. The shaft(s) have an exposed conductive sphere tip 155.

In FIG. 16, a disposable sphere electrode has a single flexible conductor cable 162, for example 2 meters in length, that terminates with a safety plug 161 at its one end and with a conductive sphere 163 at its other end.

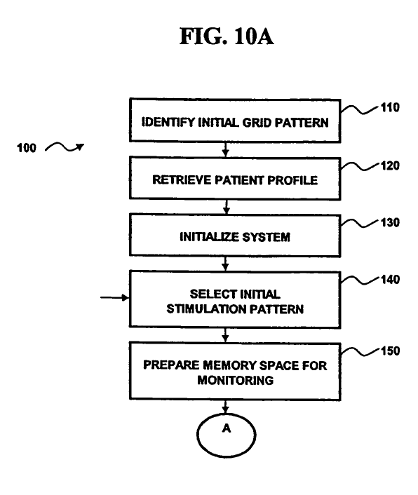

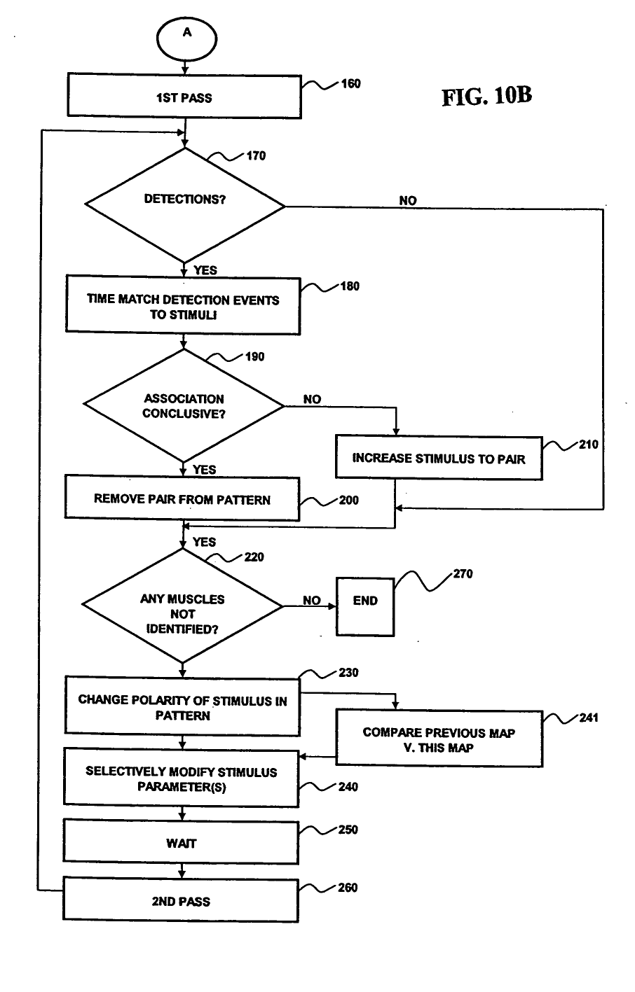

An exemplary method 100 implementing cortical mapping is now described with reference to FIGS. 10A-10B. A setup menu is first displayed on LCD 31 when turning on or resetting power to mapping stimulator 10, on a computer screen by selection of a pull-down menu bar from an attached PC, or by use of front panel buttons 52, 69 according to a firmware routine operating in communication with CPU 27. At step 110, an initial grid pattern is displayed on LCD 31 as detected, for example, by polling the output ports 26 with a current loop routine or similar resistance/continuity check to determine the type and configuration of subdural electrodes 21 presently connected. For example, a 4×5 grid may be identified on LCD 31 along with a prompt asking the user whether the indicated configuration is correct. Optionally, the connection of tail(s) 73, 83 may include alignment of a keying structure so that only one type of subdural electrode grid may be attached to cortical stimulator 20.

A patient profile is loaded in step 120. The profile may include the patient's name, identifying physical features, an image file showing the patient's face, anesthetization information, prior baseline information in a form for assisting automated calibration of the EMG sensors such as prior baseline measurements for establishing a detection threshold, stimulation parameters such as those related to safety concerns (e.g., age, heart condition, likelihood of seizure, etc) and adaptable to a form for automated control of stimulation voltages and currents, procedural information, locations of monitored muscles, physiological data such as medical history, and other information. The patient profile may also contain controlling parameters for the individual pulse trains being applied sequentially, and information regarding a number and sequence of stimulation patterns in a mapping session.

The system at step 130 performs initialization routines such as self-calibration, self-test, resetting of internal system counters, monitoring of system stabilization, validation of connectivity with remote monitoring and/or control devices such as a laptop PC, etc. An initializing routine establishes a baseline for noise and calibrates the measurement setup for individual EMG sensors 41. A self-check routine is performed in cortical stimulator section 20 to assure proper functionality and operational parameters for subsequent stimulation pulse streams. The initializing routine requests the user input/select a stimulation pattern from a library of stimulation patterns and adapts the patient profile information to a selected pattern. For example, the procedural information may indicate that a first list of stimulation patterns would be appropriate, and patient anesthetization and physiological data may dictate that a particular pattern from the list be used. The profile information may also provide for delays between mapping passes due to a patient's particular susceptibilty to a total stimulation (e.g., aggregate power) within a period of time. Any initialization failure causes CPU 27 to run an error routine that displays an error code or message on LCD 31 and that disables stimulation pending correction of the initialization problem. Otherwise, when an attached PC is being used for data collection and/or mapping control, LCD 31 displays, “Self-test OK . . . Select mode.” When operation is controlled by buttons resident on cortical mapping instrument 60, LCD 31 displays, “Self-test OK . . . Press ENTER to continue.”

Next, LCD 31 and/or PC 5 displays the patient's name, the stimulation name, the stimulation pass number or sequence, and any other selected information specific to the patient. Simultaneously, at step 140, LCD 31 and/or PC 5 also displays, “Select stimulation pattern.” The pattern may represent a single pass or series of passes. The stimulation pattern is selected either manually or automatically. In a manual mode, the amplitude, shape, and number of pulses in each pulse train may be individually set, or a pulse train may be selected from a menu. The pattern may include a series of pulse trains that are each matched to a specific pair of electrodes 21, with selectable intervals between successive pulse trains. The pattern may be set to have passes be individually triggered by use of the remote control I/O 61 and/or enter button 54. The currently identified pass may be modified by selecting stimulation of only certain pairs of electrodes 21, such as when the surgeon determines that only a particular region of the cortex is of interest. Alternatively, the stimulation passes of a pattern may be set to be performed automatically. In such a case, the menu displayed on LCD 31 or PC 5 allows the user to either program time intervals between individual passes or select a pattern having predetermined intervals. In any mode of operation, ones of the electrodes 21 may be eliminated from subsequent passes when detection of EMG events is associated with the electrode(s) 21.

The pattern may be selected to automatically stimulate a first electrode pair on one side of grid 80, followed by stimulation of a second electrode pair on an opposite side of grid 80, followed by stimulation of a third pair of electrodes having an electrode in common with the first electrode pair, etc. Such an initial stimulation pattern may include a pattern for changing parameters of applied stimulus such as voltage level, etc. For example, it may be desirable to start a mapping session using low stimulation voltages during a first pass, and then automatically increase voltages used in subsequent passes according to an interactive algorithm that stops increasing/applying a stimulation voltage to a particular electrode pair or grid area when a stimulus has been matched to a detected EMG event. In this manner, a minimum voltage or current is used for cortical mapping. The initial stimulation pattern may also be based on a focus on particular grid points of most interest, pre-operative testing, a particular procedure being performed, and various other data related to the patient profile. The patient profile itself is preferably a dynamic set of data that may create new data types and refine functions, for example according to algorithms of an expert system.

In step 140, the stimulation pattern and associated parameters may be obtained by the cortical stimulator section 20 via an input/output section 24. I/O section 24 may include one or more common interfaces such as wireless (e.g., RF, optical, etc.), USB, serial, parallel, intranet, Internet, or similar technology for externally communicating with controller 23, computer(s) 5, and/or memory device(s) 30, 50. Controller 23 includes control circuitry for causing the signal generator 29 to supply the specified pulse trains to the subdural electrodes 21 via output ports 26. Computer(s) 5 may include, for example, various databases accessed over a computer network. For example, a computer 5 may be configured with a host microprocessor, random access memory (RAM), read-only memory (ROM), input/output (I/O) electronics, a clock, a display screen, and an audio output device. A host microprocessor can include a variety of available microprocessors from Intel, Motorola, or other manufacturers. A microprocessor can be single microprocessor chip, or can include multiple primary and/or co-processors. A computer system 5 can receive sensor data or a sensor signal from EMG sensors 41, monitor real-time mapping passes or receive summary map reports, and may receive other information and/or send control signals and control data, such as for controlling other peripheral devices and/or modifying simulation pattern(s) via I/O section 24.

One or more clocks are used for synchronizing and/or time-stamping stimulation events and detection events. Clocks are also used as components of computer 5 and may be a standard clock crystal or equivalent component used to provide timing to electrical signals used by components of the computer system 5. The various clocks may be integrated, although this does not imply that synchronous operation is required. In fact, asynchronous operation may utilize continuous monitoring of EMG sensors 41, and may be adapted for using various criteria for associating a particular detection event with a stimulus. On the other hand, a use of a system clock common to control of both stimulation and detection allows for precise digital filtering and various advanced detection algorithms that increase accuracy while lowering threshold stimulation levels.

At step 150, memory space in mapping stimulator 10 is allocated by controller 23 for processing and storing data for the selected stimulation patterns. Memory may be used for EMG monitoring data and associated time-stamp information, stimulation event logging, error logging, data manipulation including filtering and other computations, etc. For example, a pattern may have corresponding libraries containing function prototypes for all the functions in a library, definitions of data types, etc., and associated memory requirements that evolve as data are collected and processed. The memory requirements may also depend on the types of filtering used for detecting barely discernable muscle response signals above background noise, a number of EMG electrodes 41, stimulus repetition rate(s), length of procedure and associated number of mapping sessions, complexity of expert system, data storage requirements, real-time processing requirements, etc. For example, EMG signals may be simultaneously monitored for detection events for ten, twenty or more different muscles. When the memory allocation has been completed, LCD 31 and/or PC 5 indicate, Ready.”

A first mapping pass is performed at step 160. Signal generator 29 outputs a set of pulse trains for a first pass of a mapping session, according to the stimulation pattern and associated waveform parameters. The pulse trains may be fed to a multiplexer section of signal generator 29 for distribution of individual pulse trains to corresponding pairs of subdural electrodes 21, via output ports 26 and tail(s) 73, 83.

At step 170, EMG detection signals are analyzed to determine whether a detection event has occurred at any of the muscles being monitored. Since it is desirable to minimize a total stimulation amount, it is important to accurately detect EMG events while doing so with a minimum of stimulus. Accordingly, it is important to achieve a high signal-to-noise ratio by optimizing filtering, using high quality electrodes 41 and associated cabling, and optimizing gain and phase properties of the EMG section 40. When an EMG response has been detected, the event is time stamped or otherwise associated with a corresponding stimulus event by controller 23 in step 180. Filtering may utilize profiles of known response shapes for adjusting filter parameters and may utilize phase detectors and similar technology for dynamically adjusting filtering operations. At step 190, a relative confidence of the EMG detection event is analyzed to determine whether the event is conclusive or whether the stimulus may need to be repeated or modified. When the EMG detection event is deemed conclusive, then the controller 23 determines in step 200 which electrodes 21 or electrode pairs may be eliminated from the stimulation pattern of a subsequent pass of the mapping session. When controller 23 determines that an EMG detection event is not conclusive, the parameters for stimulating the particular electrode pair are evaluated and modified in step 210 if controller 23 determines that, for example, a higher stimulation voltage should be used in a subsequent pass. In addition, when the EMG detection event is inconclusive, detection parameters such as IIR filtering and spatial alignment may be adjusted for improving detection accuracy corresponding to a subsequent pass.

At step 220, controller 23 determines whether the current mapping session has met all its objectives, such as identifying all monitored muscles with the required degree of confidence. If so, the mapping session is terminated in a step 270. If not, the electrode 21 pairs not eliminated from subsequent patterns in step 200 have the polarities of a next applied stimulus reversed in step 230. In addition, these remaining electrode 21 pairs are analyzed in step 240 regarding whether their corresponding stimulation parameters should be modified for the next subsequent pass. In that regard, a method known as “cross correlation” may be used for both comparing a prior map with the presently existing map, and by a diagnostic process where stimulation and detection data are compared with comparable data for normal patients. For example, a cross-correlation may be formed between the patient's response curve and a normal response curve, a maximum cross-correlation may be formed for any time shift in a selected period, and/or a correlation may be based on the time shift at which the maximum correlation occurs. Selected ones of these values for particular evoked potentials may be used to compute diagnostic feature values, and a plurality of diagnostic feature values may be used to optimize filtering, to minimize cumulative stimulation by optimizing stimulation waveforms, to profile response characteristics such as latency, etc. Additional algorithms for modifying a stimulation and detection pass may include other similar processes known for expert systems. In addition, fuzzy logic algorithms may be employed for discriminating EMG detection events from signal noise, and for determining dynamic adjustments in associated filtering.

After EMG detection events have been associated with stimulation points, and the corresponding data has been processed, a waiting period between passes may be utilized in step 250. The waiting period may be a calculated period inserted between passes when the mapping stimulator is operating in fully automated mode, may be a period when a surgeon is satisfied with a present mapping and/or does not presently need mapping results, may be based on a safety of the patient such as by locking out the cortical stimulator section 20 for a period where the patient's brain recovers from prior stimulation. When the waiting period has expired, a next mapping pass is initiated in step 260. Subsequent operations and associated processing may proceed in a manner similar to that just described for the first pass.

A physician may wish to use cortical stimulator 10 in manual mode whereby she is able to adjust stimulation parameters using analog controls. A first amplitude control 34 is provided for adjusting a level of the voltage being output to the electrodes via probe output port 39. For example, an electrical connector for multi-contact medical electrodes as described in U.S. Pat. No. 6,415,168, granted to Putz and herein incorporated by reference, may be used in output port 39. Such a connector has an array of electrical conductors, such as spring-loaded ball plungers, for contacting an electrical connector for multi-contact medical electrodes with plural contact tails 83.

In addition, a property known as “facilitation,” a process of lowering the resistance of a neural pathway by repeated passage of an impulse along the same pathway, and analogous phenomena, may influence interpulse wait times and peak voltages of stimulation pulses. As explained by Kalkman et al., Anesthesiology, V 83, No. 2, August 1995, “temporal summation” may result in a first stimulation lowering the excitation threshold of the cortical neurons, thereby facilitating the initiation of neuronal discharge by a second stimulus. Each time a neuronal terminal depolarizes, sodium channels open for a period of time. After closure of the channels, the resulting excitatory postsynaptic potential decreases over ta subsequent period. A second opening of the same channels within this period results in an augmentation (temporal summation) of the excitatory postsynaptic potential (Guyton). A similar phenomenon is called “spatial summation,” the summation of excitatory postsynaptic potentials from several synaptic terminals converging on one motor neuron.

While the principles of the invention have been shown and described in connection with specific embodiments, it is to be understood that such embodiments are by way of example and are not limiting. Consequently, variations and modifications commensurate with the above teachings, and with the skill and knowledge of the relevant art, are within the scope of the present invention. The embodiments described herein are intended to illustrate best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1. A system comprising:
a plurality of subdural electrodes formed as a grid;
a cortical stimulator for stimulating individual pairs of the plurality of subdural electrodes; and
an electromyograph for detecting reaction to the stimulating.

2. A system comprising:
a plurality of subdural electrodes formed as a grid;
a cortical stimulator for stimulating individual pairs of the plurality of subdural electrodes;
an electromyograph for detecting reaction to the stimulating; and
a controller structured for associating the reaction with one of the individual pairs of the plurality of subdural electrodes.

3. The system of claim 2 wherein the electromyograph includes a plurality of sensors adapted to monitor electrical activities of a corresponding plurality of muscle areas.

4. The system of claim 3 wherein the controller is further structured for presenting a map that matches at least one of the individual pairs of subdural electrodes respectively to at least one of the plurality of sensors.

5. The system of claim 3 wherein the controller is operative to minimize individual voltages being applied by the stimulating to the individual pairs of subdural electrodes according to at least one algorithm that pragmatically increases ones of the individual voltages within a predetermined voltage range.

6. The system of claim 5 wherein the plurality of sensors are each associated with a respective starting voltage used by the at least one algorithm.

7. The system of claim 5 wherein the at least one algorithm calculates a stimulus voltage to be applied by an individual one of the plurality of subdural electrodes based on a best guess of a threshold response voltage for the type of muscle expected to be stimulated by the one subdural electrode.

8. The system of claim 2 wherein the controller is structured for causing the cortical stimulator to stimulate the individual pairs of the plurality of subdural electrodes in a stimulation pattern.

9. The system of claim 8 wherein the stimulation pattern includes a sequence of individual passes, each pass including a sequence of applying stimuli to the individual pairs, the sequence of applying stimuli including pairing individual ones of the plurality of subdural electrodes according to a predetermined pairing pattern.

10. The system of claim 9 wherein the pairing pattern includes, for each electrode, pairing the electrode with each adjacent electrode of the grid.

11. The system of claim 9 wherein the pairing pattern is based on a stimulation minimization algorithm.

12. The system of claim 11 wherein the stimulation minimization algorithm is based on prior verification of mapping data for at least one of the plurality of subdural electrodes, and eliminating verified electrodes from the pairing pattern.

13. The system of claim 2 wherein the grid is formed as a three-dimensional array.

14. The system of claim 2 wherein the controller is further structured for creating a stimulation threshold profile for a patient, the profile including a chart of individual stimulation voltages for a plurality of stimulation points, the stimulation voltages each being a minimum voltage for evoking a particular muscle response.

15. The system of claim 8 wherein the controller includes an expert system for modifying the stimulation pattern.

16. The system of claim 3 wherein the plurality of sensors comprise at least one of monopolar, bipolar, and tripolar probes.

17. A method comprising:
providing a plurality of subdural electrodes formed as a grid;
electrically stimulating individual pairs of the plurality of subdural electrodes;
detecting an electromyographic reaction to the stimulating; and
associating the reaction with one of the individual pairs of the plurality of subdural electrodes.

18. The method of claim 17 wherein the detecting includes monitoring electrical activities of a plurality of muscles using a corresponding plurality of sensors.

19. The method of claim 18 further comprising mapping a stimulated location to one of the plurality of muscles according to a mapping function.

20. The method of claim 17 further comprising creating a stimulation threshold profile for a patient, the profile including a chart of individual stimulation voltages for a plurality of stimulation points, the stimulation voltages each being a minimum voltage required to evoke a particular muscle response.

21. The method of claim 17 wherein the stimulating is performed in at least one pattern of sequential applications of voltage to the individual pairs of the subdural electrodes.

22. The method of claim 21 further comprising modifying the pattern with an expert system.

23. The method of claim 21 wherein individual voltages being applied to the individual pairs are minimized according to at least one algorithm that pragmatically increases ones of the individual voltages within a predetermined voltage range.

24. The method of claim 23 wherein the plurality of sensors are each associated with a respective starting voltage used by the at least one algorithm.

25. The method of claim 23 wherein the at least one algorithm calculates a stimulus voltage to be applied by an individual pair of the plurality of subdural electrodes based on a best guess of a threshold response voltage for the type of muscle expected to be stimulated by the subdural electrode pair.

26. The method of claim 19 wherein the plurality of muscles are grouped into a plurality of regions, and wherein, for mapping one of the regions, part of the plurality of subdural electrodes are eliminated from a stimulating pattern.

27. The method of claim 21 wherein the stimulating includes applying a first voltage to a first one of the pairs and then applying a second voltage to a second one of the pairs, wherein the first voltage is different from the second voltage.

28. The method of claim 27 wherein the first and second voltages are pulse trains.

29. The method of claim 21 wherein the polarity of a voltage being applied to one of the pairs of electrodes is reversed for successive stimulations of the one pair.

30. The method of claim 17 wherein the stimulating of individual pairs includes, for each one of the plurality of electrodes, all pairs of electrodes that include the one electrode within a predetermined electrode-distance of the one electrode.

31. The method of claim 21 wherein if a reaction event is associated with one of the pairs of electrodes during the stimulating, the one pair is eliminated from a subsequent pairing pattern of a mapping session.

32. The method of claim 21 wherein the pattern includes a sequence of individual stimulation passes, each stimulation pass including a sequence of applying stimuli to the individual pairs, the sequence of applying stimuli including pairing individual ones of the plurality of subdural electrodes according to a predetermined pairing pattern.

33. The method of claim 32 wherein the pairing pattern is based on prior verifying of mapping data for at least one of the plurality of subdural electrodes, and elimination of verified electrodes from the pairing pattern.

34. The method of claim 21 wherein the stimulating is performed in three dimensions.

35. The method of claim 19 wherein the mapping includes presenting a map.

36. The method of claim 35 further comprising comparing the map with a predetermined map and determining at least one dimensional offset therefrom for determining a shifted position of the grid.

37. The method of claim 35 further comprising guiding a resectioning of a cortex based on the map.

38. The method of claim 35 wherein the mapping includes reading a data set, the data set defining a series of electromyograph detection scans, and displaying a relationship between the data set and a scaled graphical image.

39. The method of claim 17 further comprising mapping a set of stimulated locations to corresponding ones of the plurality of muscles according to a mapping function, wherein the mapping includes reading a data set, the data set defining a series of electromyograph detection scans, and displaying a relationship between the data set and a stored map profile of a cortex.

40. The method of claim 17 wherein the stimulating includes applying electricity to individual sets of the subdural electrodes in a predetermined pattern.

41. A method comprising utilizing subdural electrodes as selectable stimulus points in a closed loop system of cortical mapping based on electromyographic detection events.

42. Apparatus for intraoperatively localizing functional areas of the brain comprising:
means for stimulating a portion of a cortex;
means for detecting a reaction to the stimulating; and
means for associating the detecting with the stimulating.
